(19) United States
(12) Patent Application Publication
(10) Pub. No.: US 2006/0052475 A1
(43) Pub. Date: Mar. 9, 2006

(54) Uv-initiated thermally cross-linked acrylate pressure-sensitive adhesive substances

(75) Inventors: Marc Husemann, Hamburg (DE); Stephan Zollner, Hamburg (DE)

Correspondence Address: NORRIS, MCLAUGHLIN & MARCUS, P.A., 875 THIRD AVE, 18TH FLOOR, NEW YORK, NY, 10022, US

(73) Assignee: Tesa AG, Hamburg (DE)

(21) Appl. No.: 10/524,828

(22) PCT Filed: Jul. 25, 2003
(86) PCT No.: PCT/EP03/08238

§ 371 Date: Aug. 23, 2005

(30) Foreign Application Priority Data
Aug. 20, 2002 (DE) 102 37 950.5

(51) Int. Cl. A61L 2/08

(52) U.S. Cl. 522/79

(57) ABSTRACT

Process for UV-initiated thermal crosslinking within a hotmelt process for the preparation of polyacrylate hotmelt adhesives, in which a polymer is produced from an acrylate-based comonomer mixture that includes monomers which carry at least one functional group capable of reacting with a photochemically generated base, with or without the addition of a catalyst, and then coating this polymer onto a backing, irradiating within the coating process or thereafter, and finally subjecting the polymer to thermal treatment, with crosslinking occurring between the polymer and the photochemically generated base. Structural crosslinking of the sheetlike pressure-sensitive adhesive can be produced if irradiation takes place through a mask.

The invention relates to a new UV-initiated thermal crosslinking of acrylate pressure-sensitive adhesives both by comprehensive and by selective irradiation.

Within the field of pressure-sensitive adhesives (PSAs), ongoing technological developments in the coating process mean that there is a progressive need for new developments. In the industry hotmelt processes with solvent-free coating technology are of increasing importance in the preparation of PSAs, since the environmental regulations are becoming ever greater and the prices of solvents continue to rise. Consequently solvents are to be eliminated as far as possible from the manufacturing operation for PSA tapes. One rational route would be the introduction of the hotmelt technology. The introduction of this technology, however, also imposes very great requirements on the PSA. Acrylate PSAs in particular are the subject of very intensive investigations aimed at improvements. For high-end industrial applications preference is given to polyacrylates, on account of their transparency and weathering stability. As well as these advantages, however, these acrylate PSAs must also meet stringent requirements in respect of shear strength and bond strength. This profile of requirements is met by polyacrylates of high molecular weight and high polarity, with subsequent, efficient crosslinking. The drawback of these polar, high-shear-strength PSAs, however, is that they are unsuited to the operation of hotmelt extrusion, since high application temperatures are required and since, moreover, the molecular weight of the polymer is reduced by shearing in the extruder. This damage significantly lowers the level of adhesive performance. The bond strength and the tack are generally low, since the glass transition temperature is relatively high, owing to the polar fractions in the adhesives. The shear strengths in particular of hotmelt-coated acrylate PSAs drop significantly in comparison to the original solvent-coated PSA. One major reason lies in the quality of crosslinking, since acrylate PSAs from solution are generally crosslinked thermally. Thermal crosslinking possesses a great advantage: low molecular weight constituents in the polymer are crosslinked as well and so thermally crosslinked PSAs exhibit a higher level of cohesion.

Acrylate hotmelts cannot be thermally crosslinked, since the very act of hotmelt processing imposes high temperatures, which would cause gelling during the operation. In general, therefore, acrylate hotmelts are UV crosslinked or crosslinked by means of electron beams (EB). Examples of UV and/or EB crosslinking are U.S. Pat. No. 5,194,455 or DE 27 43 979 or U.S. Pat. No. 5,073,611.

In U.S. Pat. No. 5,877,261 acrylate hotmelts are crosslinked with blocked polyisocyanates. The shelf life of these polymers, however, is limited. Moreover, crosslinking on the backing material requires the use of very high temperatures, which result in damage to the backing. One specific example is the drying-out of release papers; another example is the partial melting of PP or PE backing materials.

There is therefore a need for a thermal crosslinking process for acrylate hotmelt PSAs in which crosslinking is not initiated until during or after coating and so no crosslinking reactions can occur in the hotmelt operation.

It is an object of the invention to provide a polyacrylate PSA which can be concentrated to a hotmelt and can be processed from the melt, with thermal crosslinking being possible after or during the coating operation. The crosslinking reaction ought preferably to take place thermally on the backing material, and to raise the shear strength of the PSA.

This object is achieved by means of a polyacrylate pressure-sensitive adhesive as claimed in claim 1 and also by an associated process for preparing polyacrylate hotmelt pressure-sensitive adhesives as claimed in claim 5, and by the use of these products as claimed in claim 13. The subclaims relate to advantageous developments of the invention.

The invention accordingly provides polyacrylate pressure-sensitive adhesives which essentially comprise a polymer formed from

a) a comonomer mixture comprising
a1) acrylic acid and/or acrylic esters of the following formula
   CH₂═C(R¹)(COOR²),
where R¹═H or CH₃ and R² is an alkyl chain having 1-20 carbon atoms, at 55%-99% by weight, based on component (a),
a2) olefinically unsaturated monomers having functional groups, specifically in particular having hydroxyl groups, sulfonic acid groups, ester groups, ether groups, anhydride groups, epoxy groups, amide groups, amino groups, having aromatic, heteroaromatic and/or heterocyclic groups, at 0-30% by weight, based on component (a),
a3) acrylate or methacrylate having at least one functional group at 1%-15% by weight, based on component (a), which is capable of reacting with a photochemically generated base b), with or without addition of a catalyst,
   the polymer being thermally crosslinked at least partly with a base b) in a fraction of 0.01%-25% by weight, based on the overall polymer mixture.

In one very preferred version use is made for the monomers a1) of acrylic monomers, which include acrylic and methacrylic esters with alkyl groups consisting of 4 to 14 carbon atoms, preferably 4 to 9 carbon atoms. Specific examples, without wishing to be restricted by this enumeration, are n-butyl acrylate, n-pentyl acrylate, n-hexyl acrylate, n-heptyl acrylate, n-octyl acrylate, n-nonyl acrylate, lauryl acrylate, stearyl acrylate, behenyl acrylate, and their branched isomers, such as 2-ethylhexyl acrylate, for example. Further classes of compound for use, which may likewise be added in small amounts under a1), are methyl methacrylates, cyclohexyl methacrylates and isobornyl methacrylates.

In one further preferred version use is made for the monomers a2) of vinyl esters, vinyl ethers, vinyl halides, vinylidene halides and vinyl compounds having aromatic rings and heterocycles in a position. Here as well mention may be made, nonexclusively, of some examples: vinyl acetate, vinylformamide, vinylpyridine, ethyl vinyl ether, vinyl chloride, vinylidene chloride and acrylonitrile. In a further very preferred version use is made for the monomers a2) of monomers having the following functional groups: hydroxyl, carboxyl, acid amide, isocyanato or amino groups. These groups are used for controlling the adhesive performance properties, and not as functional groups for crosslinking with the base that is formed.

In one advantageous variant use is made, for a2), of acrylic monomers conforming to the following general formula 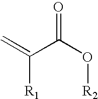
where R₁═H or CH₃ and the radical —OR₂ constitutes or comprises the functional group of the pressure-sensitive adhesive and not as a functional group for crosslinking with the base formed from b).

Particularly preferred examples of component a2) are hydroxyethyl acrylate, hydroxypropyl acrylate, hydroxyethyl methacrylate, hydroxypropyl methacrylate, allyl alcohol, maleic anhydride, itaconic anhydride, itaconic acid, acrylamide, benzyl acrylate, benzyl methacrylate, phenyl acrylate, phenyl methacrylate, t-butylphenyl acrylate, t-butylphenyl methacrylate, phenoxyethyl acrylate, phenoxyethyl methacrylate, 2-butoxyethyl methacrylate, 2-butoxyethyl acrylate, dimethylaminoethyl methacrylate, dimethylaminoethyl acrylate, diethylaminoethyl methacrylate, diethylaminoethyl acrylate, cyanoethyl methacrylate, cyanoethyl acrylate, 6-hydroxyhexyl methacrylate, N-tert-butylacrylamide, N-methylolmethacrylamide, N-(buthoxymethyl)methacrylamide, N-methylolacrylamide, N-(ethoxymethyl)acrylamide, N-isopropylacrylamide, vinylacetic acid, tetrahydrofurfuryl acrylate, β-acryloyloxypropionic acid, trichloracrylic acid, fumaric acid, crotonic acid, aconitic acid and dimethylacrylic acid, this enumeration not being conclusive.

In a further preferred version use is made for component a2) of aromatic vinyl compounds, where the aromatic nuclei may be composed preferably of C₄ to C₁₈ and may also include heteroatoms. Particularly preferred examples are styrene, 4-vinylpyridine, N-vinylphthalimide, a-methylstyrene, 3,4-dimethoxystyrene and 4-vinylbenzoic acid, this enumeration not being conclusive.

For the monomers a3) use is made of monomers which carry a functional group which is capable of reaction with the base generated from b), where appropriate with the involvement of a catalyst or of another crosslinker substance. In preferred versions use is made of comonomers having at least one carboxylic acid group, one isocyanato group or epoxide group.

In very preferred versions use is made of comonomers such as glycidyl methacrylate, acrylic acid, methacrylic acid or 2-isocyanatoethyl methacrylate.

For the polymerization the monomers are chosen such that the resulting polymers can be used as PSAs with industrial utility, particularly such that the resulting polymers possess pressure-sensitive adhesion properties in accordance with the “Handbook of Pressure Sensitive Adhesive Technology” by Donatas Satas (van Nostrand, New York 1989). For these applications the static glass transition temperature of the resulting polymer is advantageously below 15° C.

As photobase generators it is preferred to use O-acyl oximes, anilide derivates, ammonium salts or organometallic compounds which under UV irradiation liberate a base.

In one preferred version O-acyl oximes are used as photobase generators.

Depicted below are a number of preferred variants, without any wish to be restricted to these compounds.

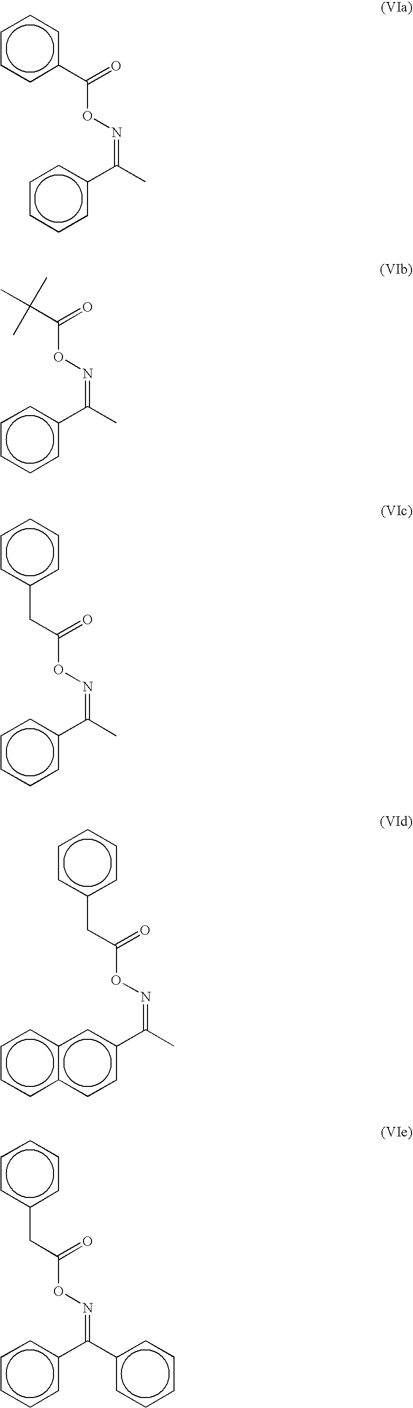

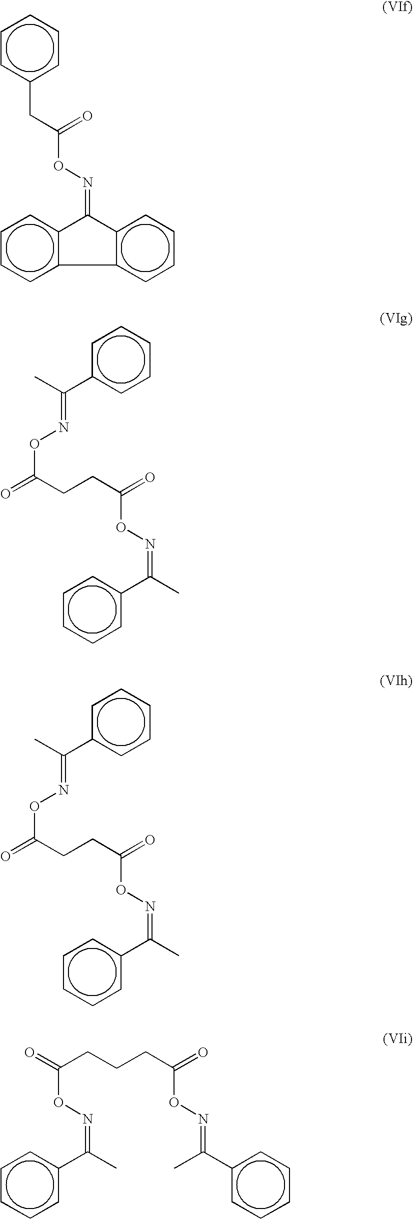

The PSAs of the invention are crosslinked by means of the base obtained from the photobase generator, in accordance with the following scheme:

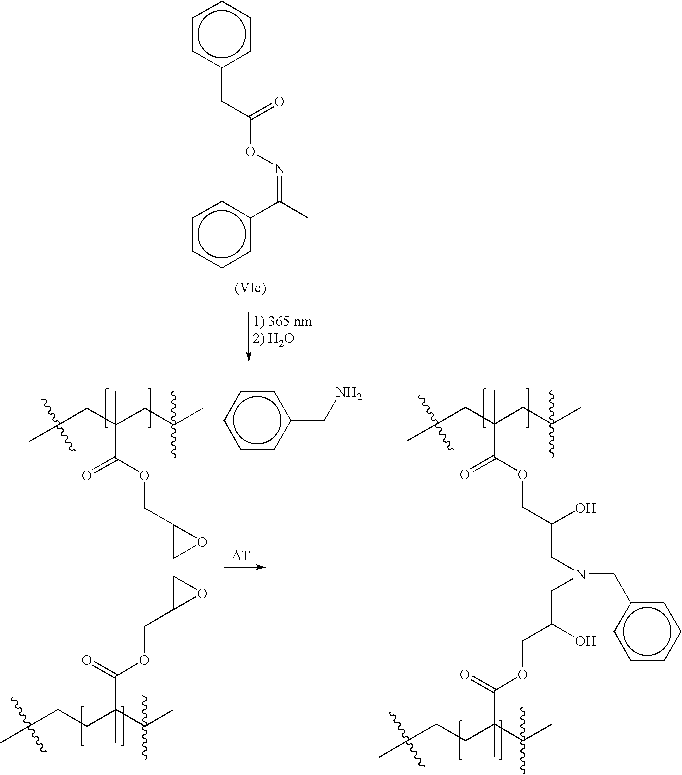

The amount of photobase generators added is guided by the amount of functions (in this case, epoxide functions) which are to be used to crosslink the acrylate PSA. In a very preferred case equimolar amounts are added to the molar fraction of epoxide-containing comonomers. In this context it is necessary to bear in mind that, by virtue of its chemical structure, a photobase generator may also liberate two molecules of amine (in this case, benzylamine). Moreover, one molecule of amine, or benzylamine, reacts with two polymers in order to effect a linking reaction. Via the amount of photobase generator it is therefore possible to vary the number of linkage sites to be produced. It is also necessary to bear in mind, with regard to the amount of photobase generator added, that the yield of the resulting amine after irradiation is likewise limited. The quantum yield of amine formation depends on the one hand on the chemical structure of the photobase generator and on the other on the polymer matrix.

A further influencing factor is the crosslinking efficiency. The benzylamine liberated is likewise able to react with two epoxide functions of a polymer chain, so that no crosslinking takes place as a result of this reaction. This influencing factor must also be borne in mind with respect to the amount of photobase generator added.

Besides the free O-acyl oximes it is also possible to introduce photobase generators by way of the polymer. In this case, in one preferred version, O-acyl oximes or similar compounds having a vinylic double bond are copolymerized into the acrylate PSA or are produced along the polymer chain. Preferred reactive groups available are acrylate, methacrylate and terminal vinyl compounds. These compounds can be employed in the sence of the monomers a2). In this particular case there is no need to add free photobase generators. In order to achieve efficient crosslinking with this method, however, it is advisable to add a crosslinking component as well. Suitability in this case is possessed by groups having a functionality of at least two and carrying a group X which is capable of reaction with the generated base. In certain particularly preferred versions hydroquinone, difunctional epoxides or difunctional carboxylic acids are added.

Formanilide derivatives as well can be added as photobase generators. In this case irradiation with UV light liberates amine compounds, which are then able to react, for example, with carboxylic acid groups of the polymer chains and hence to bring about crosslinking.

Depicted below are a number of specific examples, without any wish that this enumeration should constitute any restriction.

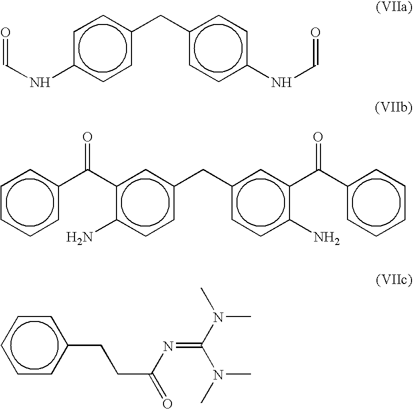

Besides the group of the anilides it is also possible to use ammonium salts as photobase generators, these salts generating free amines when irradiated with UV light. Listed below are a number of specific examples, without wishing to be restricted thereby.

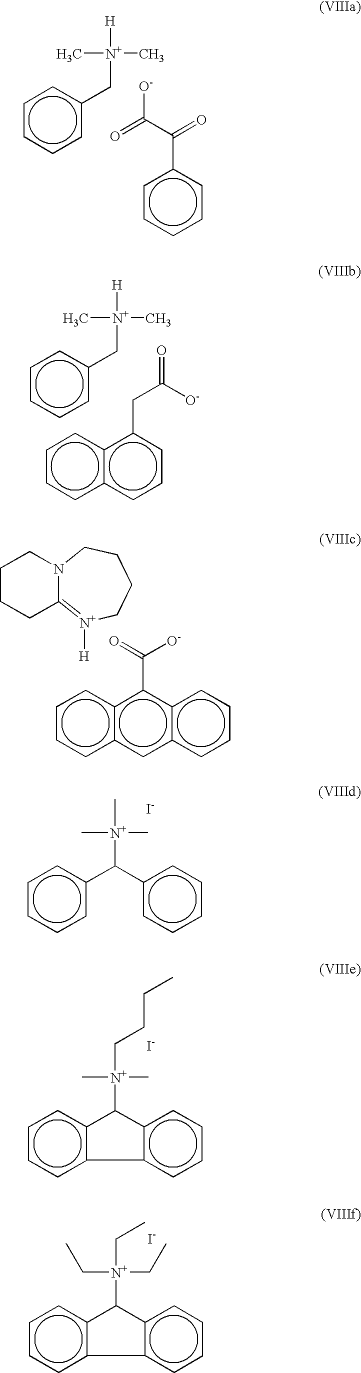

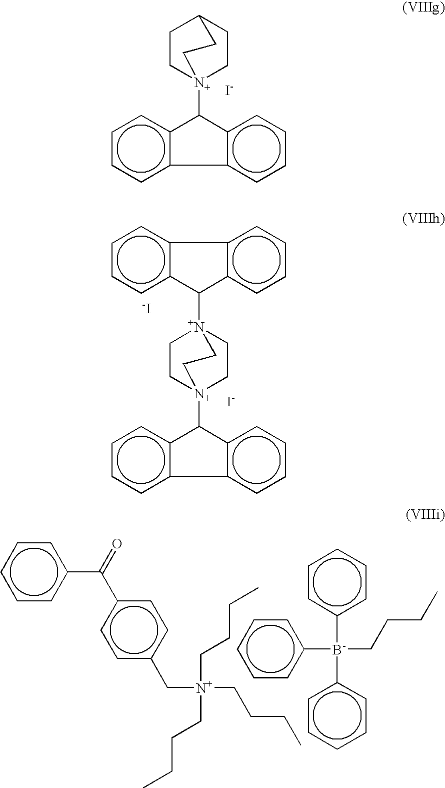

In addition to the group of the anilides and ammonium salts as photobase generators it is also possible to use photobases which liberate polyfunctional thiols after UV irradiation. A specific example is depicted below.

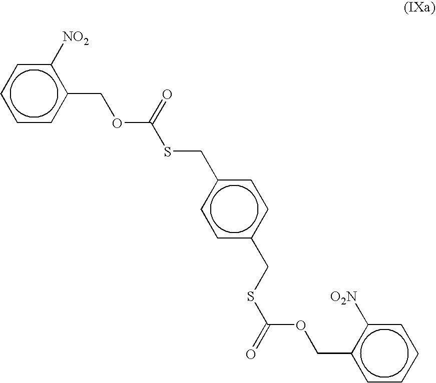

As well as the above photobase generators there exists a multiplicity of organometallic compounds which likewise liberate bases under UV irradiation.
[Co(III)(NH₂C₃H₇)₅Br]²^{⁺} (Xa)
[Co(III)(NH₃)₆]³⁺B(C₆H₅)₄⁻2B(C₆H₅)₄ (Xb)

As well as amines, however, imidazoles as well can be generated by UV irradiation using, for example, the following photobase generator.

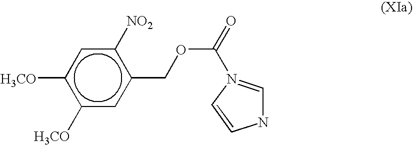

Further difunctional amines can be produced using the following compounds.

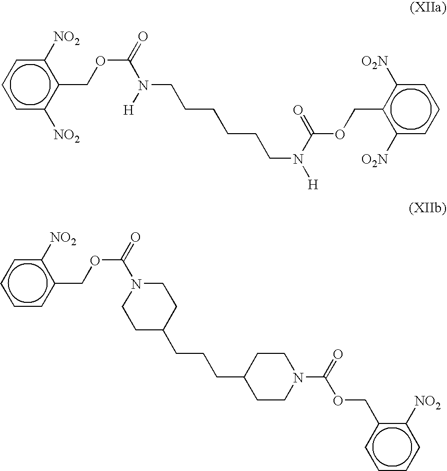

For crosslinking with UV light, in one very advantageous version of the invention, further UV-absorbing photoinitiators are added to the polyacrylate PSAs. Useful photoinitiators whose use is very beneficial are benzoin ethers, such as benzoin methyl ether and benzoin isopropyl ether, substituted acetophenones, such as 2,2-diethoxyacetophenone (available as Irgacure 651® from Ciba Geigy®), 2,2-dimethoxy-2-phenyl-1-phenylethanone, dimethoxyhydroxyacetophenone, substituted ketols, such as 2-methoxy-2-hydroxypropiophenone, aromatic sulfonyl chlorides, such as 2-naphthylsulfonyl chloride, and photoactive oximes, such as 1-phenyl-1,2-propanedione 2-(O-ethoxycarbonyl)oxime, for example. In one very preferred version benzophenone is added.

The abovementioned photoinitiators and others which can be used, and others of the Norrish I or Norrish II type, may contain the following radicals: benzophenone, aceto-phenone, benzil, benzoin, hydroxyalkylphenone, phenyl cyclohexyl ketone, anthraquinone, trimethylbenzoylphosphine oxide, methylthiophenyl morpholine ketone, amino ketone, azo benzoin, thioxanthone, hexarylbisimidazole, triazine or fluorenone, it being possible for each of these radicals additionally to be substituted by one or more halogen atoms and/or one or more alkyloxy groups and/or one or more amino groups or hydroxyl groups. A representative overview is given by Fouassier: “Photoinitiatation, Photopolymerization and Photocuring: Fundamentals and Applications”, Hanser-Verlag, Munich 1995. For supplemental information Carroy et al. in “Chemistry and Technology of UV and EB Formulation for Coatings, Inks and Paints”, Oldring (ed.), 1994, SITA, London can be consulted.

The polyacrylate PSAs of the invention are obtained by means of an associated process of the invention, in which polymers are prepared from

a) a comonomer mixture comprising
a1) acrylic acid and/or acrylic esters of the following formula
   CH₂═C(R¹)(COOR²),
where R¹═H or CH₃ and R² is an alkyl chain having 1-20 carbon atoms, at 55%-99% by weight, based on component (a),
a2) olefinically unsaturated monomers having functional groups, specifically in particular having hydroxyl groups, sulfonic acid groups, ester groups, ether groups, anhydride groups, epoxy groups, amide groups, amino groups, having aromatic, heteroaromatic and/or heterocyclic groups, at 0-30% by weight, based on component (a),
a3) acrylate or methacrylate having at least one functional group at 1%-15% by weight, based on component (a), which is capable of reacting with the base generated by b), with or without a catalyzing compound,
and b) at least one photobase generator at 0.01%-25% by weight, based. on the overall polymer mixture,
   where b) is incorporated by mixing or copolymerization and where the solvent-free polymer or the polymer substantially freed from solvent is coated with the photobase generator, in a hotmelt process, onto a backing, and during or after its coating is irradiated with UV light, thereby generating a base photochemically, and the composition is subsequently crosslinked thermally by reaction at least of component a3) with the base.

The solvent—if such is used—is preferably removed with heating under reduced pressure. In one particularly advantageous embodiment the polymer can be laid onto a film of water and subsequently transferred to the backing material, with the water preferably contributing to the crosslinking of the PSA. This is described in more detail below.

For the preparation of the polyacrylate PSAs first of all conventional radical polymerizations or controlled radical polymerizations are carried out for the purpose of preparing the polymers that are subsequently to be crosslinked. For the polymerizations which proceed by a radical mechanism it is preferred to use initiator systems which additionally include further radical initiators for the polymerization, especially thermally decomposing radical-forming azo or peroxo initiators. In principle, however, all customary initiators that are known for acrylates are suitable for this pupose. The production of C-centered radicals is described in Houben Weyl, Methoden der Organischen Chemie, Vol. E 19a, pp. 60-147. These methods are preferentially employed analogously.

Examples of radical sources are peroxides, hydroperoxides and azo compounds; as a number of nonexclusive examples of typical radical initiators mention may be made here of potassium peroxodisulfate, dibenzoyl peroxide, cumene hydroperoxide, cyclohexanone peroxide, di-t-butyl peroxide, azodiisobutyronitrile, cyclohexylsulfonyl acetyl peroxide, diisopropyl percarbonate, t-butyl peroctoate and benzpinacol. In one very preferred version a radical initiator used is 1,1′-azobis(cyclohexanecarbononitrile) (Vazo 88™ from DuPont).

The average molecular weights Mₙ of the PSAs formed in the controlled radical polymerization are chosen such that they are situated within a range from 20 000 and 2 000 000 g/mol. Specifically for further use as hotmelt PSAs, PSAs having molecular weights Mₙ of from 100 000 to 500 000 g/mol are preferred. The average molecular weight is determined by way of size exclusion chromatography (SEC) or matrix-assisted laser desorption/ionization—mass spectrometry (MALDI-MS).

The polymerization may be conducted in bulk, in the presence of an organic solvent, in the presence of water, or in mixtures of organic solvents and water. The aim is to minimize the amount of solvent used. Suitable organic solvents or mixtures of solvents are pure alkanes (hexane, heptane, octane, isooctane), aromatic hydrocarbons (benzene, toluene, xylene), esters (ethyl acetate, propyl acetate, butyl acetate or hexyl acetate), halogenated hydrocarbons (chlorobenzene), alkanols (methanol, ethanol, ethylene glycol, ethylene glycol monomethyl ether) and ethers (diethyl ether, dibutyl ether) or mixtures thereof.

A water-miscible or hydrophilic cosolvent may be added to the aqueous polymerization reactions in order to ensure that in the course of monomer conversion the reaction mixture is in the form of a homogeneous phase. Cosolvents which can be used with advantage for the present invention are selected from the following group, consisting of aliphatic alcohols, glycols, ethers, glycol ethers, pyrrolidines, N-alkylpyrrolidinones, N-alkylpyrrolidones, polyethylene glycols, polypropylene glycols, amides, carboxylic acids and salts thereof, esters, organic sulfides, sulfoxides, sulfones, alcohol derivatives, hydroxy ether derivatives, amino alcohols, ketones and the like, and also derivatives and mixtures thereof.

Depending on conversion and temperature, the polymerization time amounts to between 4 and 72 hours. The higher the reaction temperature that can be chosen, in other words the higher the thermal stability of the reaction mixture, the lower the reaction time that can be chosen.

For initiating the polymerization the introduction of heat is essential for the thermally decomposing initiators. For the thermally decomposing initiators the polymerization can be initiated by heating at 50-160° C., depending on initiator type.

For the preparation of polyacrylate PSAs having a narrow molecular weight distribution suitability is also possessed by controlled radical polymerization methods. In that case it is preferred, for the purpose of the polymerization, to use a control reagent of the general formula: 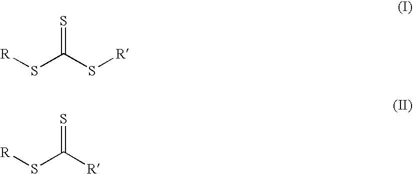
in which

R and R′, chosen independently of one another or identical, represent
branched and unbranched C₁- to C₁₈ alkyl radicals, C₃- to C₁₈ alkenyl radicals or C₃- to C₁₈ alkynyl radicals;
H or C₁- to C₁₈ alkoxy;
C₁- to C₁₈ alkyl radicals; C₃- to C₁₈ alkenyl radicals; C₃- to C₁₈ alkynyl radicals that are substituted by at least one OH group or halogen atom or silyl ether;
C₂-C₁₈ heteroalkyl radicals having at least one oxygen atom and/or one NR′ group in the carbon chain;
C₁-C₁₈ alkyl radicals, C₃-C₁₈ alkenyl radicals or C₃-C₁₈ alkynyl radicals substituted by at least one ester group, amine group, carbonate group, cyano, isocyanto and/or epoxide group and/or by sulfur;
C₃-C₁₂ cycloalkyl radicals;
C₆-C₁₈ aryl radicals or benzyl radicals;
hydrogen.

Control reagents of type (I) are composed, in one more-preferred version, of the following, further-restricted compound:

halogens in this case are preferably F, Cl, Br or I, more preferably Cl and Br. Suitable alkyl, alkenyl and alkynyl radicals in the various substituents include outstandingly not only linear chains but also branched chains.

Examples of alkyl radicals which contain 1 to 18 carbon atoms are methyl, ethyl, propyl, isopropyl, butyl, isobutyl, t-butyl, pentyl, 2-pentyl, hexyl, heptyl, octyl, 2-ethylhexyl, t-octyl, nonyl, decyl, undecyl, tridecyl, tetradecyl, hexadecyl and octadecyl.

Examples of alkenyl radicals having 3 to 18 carbon atoms are propenyl, 2-butenyl, 3-butenyl, isobutenyl, n-2,4-pentadienyl, 3-methyl-2-butenyl, n-2-octenyl, n-2-dodecenyl, isododecenyl and oleyl.

Examples of alkynyl having 3 to 18 carbon atoms are propynyl, 2-butynyl, 3-butynyl, n-2-octynyl and n-2-octadecynyl.

Examples of hydroxy-substituted alkyl radicals are hydroxypropyl, hydroxybutyl or hydroxyhexyl.

Examples of halogen-substituted alkyl radicals are dichlorobutyl, monobromobutyl or trichlorohexyl.

One suitable C₂-C₁₈ heteroalkyl radical having at least one oxygen atom in the carbon chain is for example —CH₂—CH₂—O—CH₂—CH₃.

Examples of C₃-C₁₂ cycloalkyl radicals include cyclopropyl, cyclopentyl, cyclohexyl or trimethylcyclohexyl.

Examples of C₆-C₁₈ aryl radicals include phenyl, naphthyl, benzyl, 4-tert-butylbenzyl or further substituted phenyl, such as ethyl, toluene, xylene, mesitylene, isopropylbenzene, dichlorobenzene or bromotoluene.

The listings above serve only as examples of the respective groups of compounds, and possess no claim to completeness.

Also suitable are compounds of the following types 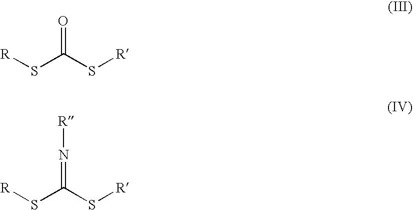
where R″ may comprise either the radicals R or R′.

In the case of the conventional RAFT process polymerization is generally carried out only up to low conversions (WO 98/01478 A1) in order to produce very narrow molecular weight distributions. As a result of the low conversions, however, these polymers cannot be used as PSAs and in particular not as hotmelt PSAs, since the high fraction of residual monomers adversely affects the adhesive performance properties; the residual monomers contaminate the solvent recyclate in the concentration operation; and the corresponding self-adhesive tapes would exhibit very high outgassing. In order to circumvent this drawback of low conversions, the polymerization in one particularly preferred version is initiated two or more times.

As a further controlled radical polymerization method it is possible to carry out nitroxide-controlled polymerizations. For radical stabilization, in a favorable procedure, use is made of nitroxides of type (Va) or (Vb): 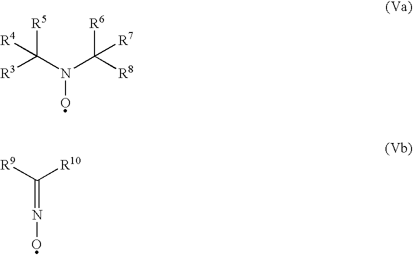
where R³, R⁴, R⁵, R⁶, R⁷, R⁸, R⁹ and R¹⁰ independently of one another denote the following compounds or atoms:
i) halides, such as chlorine, bromine or iodine, for example,
ii) linear, branched, cyclic and heterocyclic hydrocarbons having 1 to 20 carbon atoms, which may be saturated, unsaturated or aromatic,
iii) esters —COOR¹¹, alkoxides —OR¹² and/or phosphonates —PO(OR¹³)₂, where R¹¹, R¹² or R¹³ stand for radicals from group ii).

Compounds of the (Va) or (Vb) may also be attached to polymer chains of any kind (primarily such that at least one of the abovementioned radicals constitutes a polymer chain of this kind) and may thus be used, for example, as macroradicals or macroregulators for synthesizing the block copolymers.

Controlled regulators become more preferred for the polymerization of compounds of the type:
2,2,5,5-tetramethyl-1-pyrrolidinyloxyl (PROXYL), 3-carbamoyl-PROXYL, 2,2-dimethyl-4,5-cyclohexyl-PROXYL, 3-oxo-PROXYL, 3-hydroxylimine-PROXYL, 3-aminomethyl-PROXYL, 3-methoxy-PROXYL, 3-t-butyl-PROXYL, 3,4-di-t-butyl-PROXYL
2,2,6,6-tetramethyl-1-piperidinyloxy pyrrolidinyloxyl (TEMPO), 4-benzoyloxy-TEMPO, 4-methoxy-TEMPO, 4-chloro-TEMPO, 4-hydroxy-TEMPO, 4-oxo-TEMPO, 4-amino-TEMPO, 2,2,6,6-tetraethyl-1-piperidinyloxyl, 2,2,6-trimethyl-6-ethyl-1-piperidinyloxyl
N-tert-butyl 1-phenyl-2-methylpropyl nitroxide
N-tert-butyl 1-(2-naphthyl)-2-methylpropyl nitroxide
N-tert-butyl 1-diethylphosphono-2,2-dimethylpropyl nitroxide
N-tert-butyl 1-dibenzylphosphono-2,2-dimethylpropyl nitroxide
N-(1-phenyl-2-methyl propyl) 1-diethylphosphono-1-methylethyl nitroxide
di-t-butyl nitroxide
diphenyl nitroxide
t-butyl t-amyl nitroxide

U.S. Pat. No. 4,581,429 A itself discloses a controlled-growth radical polymerization process which uses as its initiator a compound of the formula R′R″N—O—Y, in which Y is a free radical species which is able to polymerize unsaturated monomers. The reactions, however, generally have low conversions. A particular problem is the polymerization of acrylates, which proceeds only to very low yields and molar masses. WO 98/13392 A1 describes open-chain alkoxyamine compounds which have a symmetrical substitution pattern. EP 735 052 A1 discloses a process for preparing thermoplastic elastomers having narrow molar mass distributions. WO 96/24620 A1 describes a polymerization process in which very specific radical compounds, such as phosphorus-containing nitroxides based on imidazolidine, for example, are used. WO 98/44008 A1 discloses specific nitroxyls based on morpholines, piperazinones and piperazinediones. DE 199 49 352 A1 describes heterocyclic alkoxyamines as regulators in controlled-growth radical polymerizations. Corresponding further developments of the alkoxyamines or of the corresponding free nitroxides improve the efficiency for the preparation of polyacrylates (Hawker, contribution to the National Meeting of the American Chemical Society, Spring 1997; Husemann, contribution to the IUPAC World Polymer Meeting 1998, Gold Coast).

As a further control polymerization it is possible with advantage to synthesize the polyacrylate PSAs using atom transfer radical polymerization (ATRP), in which case preferred initiators used are monofunctional or difunctional secondary or tertiary halides and the halide(s) is (are) abstracted using complexes of Cu, Ni, Fe, Pd, Pt, Ru, Os, Rh, Co, Ir, Ag or Au (EP 0 824 111 A1; EP 826 698A1; EP 824 110A1; EP 841 346A1; EP 850 957 A1). The various possibilities of ATRP are further described in U.S. Pat. No. 5,945,491 A, U.S. Pat. No. 5,854,364 A and U.S. Pat. No. 5,789,487 A.

For further development it is possible to admix resins to the polyacrylate PSAs. Tackifying resins for addition that can be used include, without exception, all of the tackifier resins that are already known and are described in the literature. Representatives that may be mentioned include pinene resins, indene resins and rosins, their disproportionated, hydrogenated, polymerized and/or esterified derivatives and salts, the aliphatic and aromatic hydrocarbon resins, terpene resins and terpene-phenolic resins and also C5, C9 and other hydrocarbon resins. Any desired combinations of these and further resins may be used in order to adjust the properties of the resultant adhesive in accordance with what is desired. Generally speaking it is possible to use any resins which are compatible with (soluble in) the corresponding polyacrylate, reference being made in particular to all aliphatic, aromatic and alkylaromatic hydrocarbon resins, hydrocarbon resins based on single monomers, hydrogenated hydrocarbon resins, functional hydrocarbon resins, and natural resins. Express reference is made to the depiction of the state of the art in the “Handbook of Pressure Sensitive Adhesive Technology” by Donatas Satas (van Nostrand, 1989).

Additionally it is possible optionally to add plasticizers, fillers (e.g., fibers, carbon black, zinc oxide, titanium dioxide, chalk, solid or hollow glass spheres, microspheres of other materials, silica, silicates), nucleators, expandants, compounding agents and/or aging inhibitors, in the form for example of primary and secondary antioxidants or in the form of light stabilizers.

The polyacrylate PSAs can be applied from solution or from the melt to the backing material. For application from the melt the solvent is preferably stripped off in a concentrating extruder under reduced pressure, for which purpose use may be made, for example, of single-screw or twin-screw extruders, which preferably distill off the solvent in identical or different vacuum stages and possess a feed preheater.

In one preferred version of the invention—especially if water is needed as the catalyst or for crosslinking—the PSAs are deposited on a film of water. In one preferred version of the invention the film of water is located on a metal roll.

The metal roll may be equipped with an effective cooling system, in order to transport away the considerable quantities of heat. For certain cases, however, it may also be of advantage to heat the film of water by means of a heating system.

To prevent corrosion it is commonly coated with a protective coat. This coat is preferably selected such that it is effectively wetted by the contact medium. Generally the surface is conductive. It may also be more favorable, however, to coat it with one or more coats of insulating or semiconducting material.

Additionally the metal roll may be macroscopically smooth or may have a slightly textured surface. It has been found appropriate for it to have a surface texture, in particular a surface roughening. This allows wetting by the contact medium to take place more effectively.

The process proceeds to particularly good effect if the metal roll is temperature-controllable, preferably in a range of −10 C to 200 C, very preferably from 5 C to 70 C.

As the contact liquid it is advantageous to use water. In a further variant, which is outstandingly suitable for the purposes of the invention, substances are added to the water contact medium that are at least partially soluble therein. For a water contact medium, appropriate additives include, for example, alkyl alcohols such as ethanol, propanol, butanol and hexanol, without wishing to be restricted in the selection of alcohols as a result of these examples. Also highly advantageous are, in particular, longer-chain alcohols, polyglycols, ketones, amines, carboxylates, sulfonates and the like.

It has proven very advantageous to use as the contact medium a liquid which if desired acquires additives for additional functions. These functions include the increase of surface wetting.

A reduction in the surface tension may also be achieved by adding small amounts of nonionic and/or anionic and/or cationic surfactants to the contact medium. The most simple way of achieving this is by using commercial washing compositions or soap solutions, preferably in a concentration of a few g/l in water, as contact medium. Particularly suitable compounds are specific surfactants which can be used even at a low concentration. Examples thereof that may be mentioned include sulfonium surfactants (e.g. β-di(hydroxyalkyl)sulfonium salt), and also, for example, ethoxylated nonylphenyl-sulfonic acid ammonium salts. Here particular reference may be made to the state of the art under “surfactants” in Ullmann's Encyclopedia of Industrial Chemistry, Sixth Edition, 2000 Electronic Release, Wiley-VCH, Weinheim 2000.

Where water is used as the contact medium it is possible to proceed outstandingly by running a second roller, advantageously having a wettable or absorbent surface, through a bath containing the contact medium, said roller then becoming wetted by or impregnated with the contact medium and applying a film of said contact medium by contact with the chill roll.

In one very preferred version of the invention the pressure-sensitive adhesive is irradiated with UV light directly on the film of water of the metal roll.

In a procedure which is advantageous for the process operation takes place by means of brief ultraviolet irradiation within a wavelength range from 200 to 400 nm, depending on the photobase generator used, using in particular high-pressure or medium-pressure mercury lamps with an output of from 80 to 240 W/cm. The irradiation intensity is adapted to the respective quantum yield of the photobase generator. After the operation of coating on the film of water and UV irradiation, the polyacrylate PSA is transferred from the film of water to a backing material.

Backing materials used for the PSA, such as for PSA tapes, for example, are the materials that are customary and familiar to the skilled worker, such as films (polyesters, PET, PE, PP, BOPP, PVC), nonwovens, foams, woven fabrics and woven films, and also release paper (glassine, HDPE, LDPE). This enumeration is not conclusive.

The guided supply of the backing, i.e., for example, of the release-coated materials such as papers, films or nonwovens, is advantageously performed under a certain contact pressure.

After the operation of coating the adhesive on the backing the PSA tape, in one preferred version, is further irradiated with UV light. In one advantageous variant this procedure takes place by means of brief ultraviolet radiation in a wavelength range from 200 to 400 nm, depending on the photobase generator used, in particular using high-pressure or medium-pressure mercury lamps with an output of from 80 to 240 W/cm. The intensity of irradiation is adapted to the respective quantum yield of the photobase generator.

It can be of great advantage for the process, especially if water is required as catalyst or for crosslinking, if the PSA tape is in contact with water during or after UV crosslinking. In one variant the PSA tape is run through a water bath, while in another variant the PSA tape is guided through a controlled-climate chamber featuring high air humidity. It is also possible to store the PSA tapes in a controlled-climate chamber featuring high air humidity.

In one very preferred version of the process the PSA tape is heated again after it has been UV-irradiated and moistened by means of water. In one very preferred version this can be done in a drying tunnel. To initiate the crosslinking reaction the PSA tape is heated to at least 80° C., in one very preferred variant to at least 100° C. Heating can be carried out using different sources, all of which are able to emit heat. In one very preferred version use is made of IR lamps. In very simple and practical versions, hot-air fans or other heat machines which generate by means of electrical energy are also suitable, however.

In principle it is also possible to subject the polyacrylate PSA to crosslinking with electron beams as well. Typical irradiation equipment which may be employed includes linear cathode systems, scanner systems and segmented cathode systems, where the equipment in question comprises electron beam accelerators. A detailed description of the state of the art and the most important process parameters can be found in Skelhorne, Electron Beam Processing, in Chemistry and Technology of UV and EB formulation for Coatings, Inks and Paints, Vol. 1, 1991, SITA, London. The typical acceleration voltages are situated in the range between 50 kV and 500 kV, preferably 80 kV and 300 kV. The irradiation doses employed range between 5 to 150 kGy, in particular between 20 and 100 kGy.

In a further version according to the invention the polyacrylate PSA is in principle subjected only to structured irradiation. A feature of this process for producing structured polyacrylates by means of structured crosslinking of polyacrylate mixtures is that the base polymer mixture is irradiated with ultraviolet light such that only particular regions of the polymer mixture are exposed to UV radiation.

The process for production can be conducted in particular such that the base polymer mixture is irradiated with ultraviolet light through a perforated mask in such a way that only particular regions of the polymer mixture are exposed to UV radiation.

Alternatively the structuring of the polymer mixture to be cured can be achieved by using, instead of the perforated mask, a film whose surface area includes regions of different UV-light transparency, so that certain regions of the polymer mixture are exposed to different intensities of UV radiation.

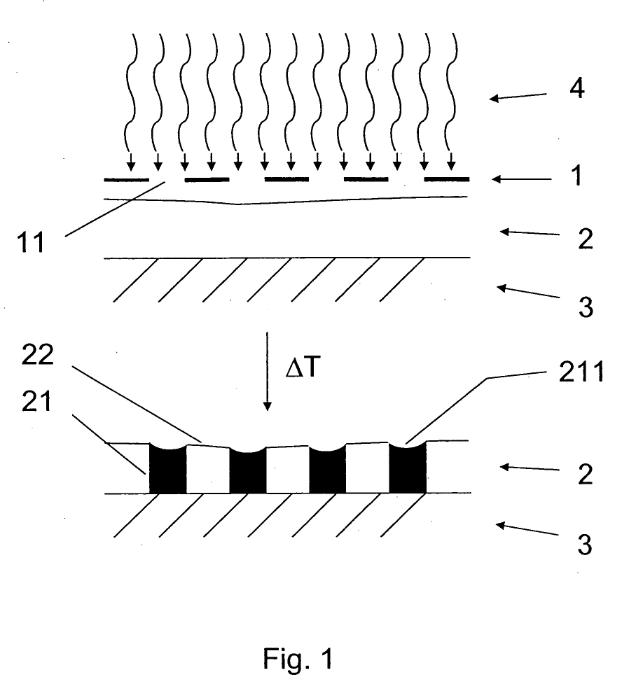

The process outlined above is illustrated below with reference to FIG. 1

FIG. 1: Diagrammatic representation of a structured crosslinking by means of UV irradiation through a perforated mask.

FIG. 1 depicts the irradiation of the acrylate composition (2) through a perforated mask (1), the acrylate composition (2) being located on the backing (3). According to the main claim the acrylate composition (1) is admixed with a photobase generator, which as a result of UV light (4) and water, and at high temperatures (ΔT), performs partial crosslinking of epoxide groups of the polymer (2). The ultraviolet rays (4) are able to penetrate the mask (1) only in the region of the perforations (11), so that following irradiation the resulting situation is that depicted in the bottom part of the figure: the PSA (2) has hard segments of high crosslinking (21) and also uncrosslinked, soft segments (22).

The polymer chains at the edges of the hard regions reach into the soft regions, and so the hard regions, which are of inherently high viscosity, are linked to the soft regions and therefore hinder these latter regions in terms of their mobility, thereby raising the structural strength of the adhesive. These hard segments, moreover, increase the cohesion of the PSA. In contrast, the soft segments (22) result in readier flow of the adhesive on the substrate and, accordingly, increase the bond strength and the tack. A large influence on the adhesive performance properties is exerted by the percentage fraction of the irradiated area and also by the size of the segments produced.

The invention further comprises the use of the polyacrylate as a pressure-sensitive adhesive, in particular its use as a pressure-sensitive adhesive for an adhesive tape, in which case the pressure-sensitive acrylate adhesive is present in the form of a one-sided or double-sided film on a backing sheet.

### EXAMPLES

The following exemplary experiments are intended to illustrate the content of the invention, without any intention that the invention should be unnecessarily restricted as a result of the choice of the examples.

Test Methods

The test methods set out below were used to characterize the polyacrylate compositions and their crosslinked products:

Shear Strength (Test A1, A2)

A strip of the adhesive tape 13 mm wide was applied to a smooth steel surface which had been cleaned. The area of application was 20 mm×13 mm (length×width). The following procedure was then adopted:

Test A1: at room temperature a 1 kg weight was attached to the adhesive tape and the time taken for the weight to fall was recorded.

Test A2: at 70° C. a 0.5 kg weight was attached to the adhesive tape and the time taken for the weight to fall was recorded.

The shear withstand times recorded are each reported in minutes, and correspond to the average of three measurements.

180° Bond Strength Test (Test B)

A strip 20 mm wide of an acrylate PSA applied as a layer on polyester was applied to steel plates which had been cleaned twice with acetone and once with isopropanol. The PSA strip was pressed onto the substrate twice using a 2 kg weight. Immediately thereafter the adhesive tape was peeled from the subsrate at an angle of 180° and at 300 mm/min, and the force required to achieve this was recorded. All measurements were conducted at room temperature.

The results are reported in N/cm and have been averaged from three measurements.

Determination of the Gel Fration (Test C)

After careful drying, the solvent-free samples of adhesive are welded into a pouch made of polyethylene nonwoven (Tyvek web). The difference in the sample weights before and after extraction using toluene determines the gel index, which is the weight fraction of polymer that is not soluble in toluene.

Implementation of the Hotmelt process in a Recording Extruder:

The shearing and thermal working of the acrylate hotmelts was carried out using the Rheomix 610p recording extruder from Haake. The drive unit available was the Rheocord RC 300p. The apparatus was controlled using the PolyLab System software. The extruder was charged in each case with 52 g of pure acrylate PSA (˜80% fill level). The experiments were conducted with a kneading temperature of 120° C., a rotary speed of 60 rpm and a kneading time of 6 hours. Thereafter the specimens were dissolved again, if possible, and the gel index was determined in accordance with test C.

UV Irradiation

UV irradiation was carried out using a UV unit from Eltosch. The unit is equipped with a medium-pressure Hg UV lamp having an intensity of 120 W/cm. The swatches were each run through the unit at a speed of 20 m/min, the specimens being irradiated in two or more passes in order to increase the radiation dose. The UV dose was measured using the Power-Puck from Eltosch. The dose of one irradiation pass amounted to about 140 mJ/cm² in the UV-B region and 25 mJ/cm² in the UV-C region.

Photobase Generators:

Bis[[(2-nitrobenzyl)oxy]carbonyl]hexane-1,6-diamine (XIIa) was prepared according to instructions in Journal of Polymer Science: Part A: Polymer Chemistry, Vol. 31, 3013-3020 (1993).

4,4′-[Bis[[(2-nitrobenzyl)oxy]carbonyl]trimethylene]dipiperidine (XIIb) was synthesized according to instructions from Macromolecules 1997, 30, pp. 1304-1310.

Regulator:

Bis-2,2′-phenylethyl thiocarbonate is synthesized starting from 2-phenylethyl bromide using carbon disulfide and sodium hydroxide in accordance with instructions from Synth. Communications 18(13), pp. 1531-1536, 1988. Yield after distillation: 72%. ¹H-NMR (CDCl₃) δ (ppm): 7.20-7.40 (m, 10 H), 1.53, 1.59 (2×d, 6 H), 3.71, 381 (2×m, 2 H).

### Example 1

A 2 L glass reactor conventional for radical polymerizations was charged with 40 g of acrylic acid, 360 g of 2-ethylhexyl acrylate, 0.55 g of bis-2,2′-phenylethyl thiocarbonate and 170 g of acetone. After nitrogen gas had been passed through the reactor for 45 minutes, with stirring, the reactor was heated to 58° C. and 0.3 g of azoisobutyronitrile (AIBN, Vazo 64™, DuPont) was added. Subsequently the external heating bath was heated to 75° C. and the reaction was carried out constantly at this external temperature. After a reaction time of 1 h a further 0.3 g of AIBN was added. Dilution took place after 4 h and 8 h, using 100 g of acetone each time. After a reaction time of 48 h the reaction was terminated and cooling took place to room temperature.

Subsequently the polymer was blended in solution with 5% by weight of bis[[(2-nitro-benzyl)oxy]carbonyl]hexane-1,6-diamine and then concentrated in a vacuum drying oven at 80° C. and a pressure of 10 torr. Subsequently the hotmelt process was carried out in the recording extruder. For crosslinking, the acrylate hotmelt was coated at 50 g/m² onto a PET film, using a heatable laboratory roll coater, irradiated with UV light in a number of passes, in accordance with the method described above, and then conditioned at 140° C. for 20 minutes. Finally the specimens were tested according to methods A and B.

### Example 2

The procedure of Example 1 was repeated. The photobase generator added was 5% by weight of 4,4′-[bis[[(2-nitrobenzyl)oxy]carbonyl]trimethylene]dipiperidine.

### Example 3

A 2 L glass reactor conventional for radical polymerizations was charged with 30 g of acrylic acid, 100 g of n-butyl acrylate, 270 g of 2-ethylhexyl acrylate, 0.55 g of bis-2,2′-phenylethyl thiocarbonate and 170 g of acetone. After nitrogen gas had been passed through the reactor for 45 minutes, with stirring, the reactor was heated to 58° C. and 0.3 g of azoisobutyronitrile (AIBN, Vazo 64™, DuPont) was added. Subsequently the external heating bath was heated to 75° C. and the reaction was carried out constantly at this external temperature. After a reaction time of 1 h a further 0.3 g of AIBN was added. Dilution took place after 4 h and 8 h, using 100 g of acetone each time. After a reaction time of 48 h the reaction was terminated and cooling took place to room temperature.

Subsequently the polymer was blended in solution with 3% by weight of bis[[(2-nitro-benzyl)oxy]carbonyl]hexane-1,6-diamine and then concentrated in a vacuum drying oven at 80° C. and a pressure of 10 torr. Subsequently the hotmelt process was carried out in the recording extruder. For crosslinking, the acrylate hotmelt was coated at 50 g/m² onto a PET film, using a heatable laboratory roll coater, irradiated with UV light in a number of passes, in accordance with the method described above, and then conditioned at 140° C. for 20 minutes. Finally the specimens were tested according to methods A and B.

### Example 4

A 2 L glass reactor conventional for radical polymerizations was charged with 8 g of glycidyl methacrylate, 100 g of isobornyl acrylate, 292 g of 2-ethylhexyl acrylate, 0.55 g of bis-2,2′-phenylethyl thiocarbonate and 170 g of acetone: special-boiling-point spirit 60/95 (1:1). After nitrogen gas had been passed through the reactor for 45 minutes, with stirring, the reactor was heated to 58° C. and 0.3 g of azoisobutyronitrile (AIBN, Vazo 64™, DuPont) was added. Subsequently the external heating bath was heated to 75° C. and the reaction was carried out constantly at this external temperature. After a reaction time of 1 h a further 0.3 g of AIBN was added. Dilution took place after 4 h and 8 h, using 100 g of spirit in each case. After a reaction time of 48 h the reaction was terminated and cooling took place to room temperature.

Subsequently the polymer was blended in solution with 3% by weight of bis[[(2-nitro-benzyl)oxy]carbonyl]hexane-1,6-diamine and then concentrated in a vacuum drying oven at 80° C. and a pressure of 10 torr. Subsequently the hotmelt process was carried out in the recording extruder. For crosslinking, the acrylate hotmelt was coated at 50 g/m² onto a PET film, using a heatable laboratory roll coater, the specimen was stored at 60° C. and 98% humidity for one day, irradiated with UV light in a number of passes, in accordance with the method described above, and then conditioned at 140° C. for 20 minutes. Finally the specimens were tested according to methods A and B.

### Example 5

A 2 L glass reactor conventional for radical polymerizations was charged with 12 g of 2-isocyanatoethyl methacrylate, 100 g of isobornyl acrylate, 288 g of 2-ethylhexyl acrylate, 0.55 g of bis-2,2′-phenylethyl thiocarbonate and 170 g of acetone: special-boiling-point spirit 60/95 (1:1). After nitrogen gas had been passed through the reactor for 45 minutes, with stirring, the reactor was heated to 58° C. and 0.3.g of azoisobutyronitrile (AIBN, Vazo 64™, DuPont) was added. Subsequently the external heating bath was heated to 75° C. and the reaction was carried out constantly at this external temperature. After a reaction time of 1 h a further 0.3 g of AIBN was added. Dilution took place after 4 h and 8 h, using 100 g of spirit. After a reaction time of 48 h the reaction was terminated and cooling took place to room temperature.

Subsequently the polymer was blended in solution with 4% by weight of bis[[(2-nitro-benzyl)oxy]carbonyl]hexane-1,6-diamine and then concentrated in a vacuum drying oven at 80° C. and a pressure of 10 torr. Subsequently the hotmelt process was carried out in the recording extruder. For crosslinking, the acrylate hotmelt was coated at 50 g/m² onto a PET film, using a heatable laboratory roll coater, irradiated with UV light in a number of passes, in accordance with the method described above, and then conditioned at 140° C. for 30 minutes. Finally the specimens were tested according to methods A and B.

### Example 6

A 2 L glass reactor conventional for radical polymerizations was charged with 28 g of acrylic acid, 80 g of methyl acrylate, 292 g of 2-ethylhexyl acrylate, 0.40 g of bis-2,2′-phenylethyl thiocarbonate and 170 g of acetone. After nitrogen gas had been passed through the reactor for 45 minutes, with stirring, the reactor was heated to 58° C. and 0.3 g of azoisobutyronitrile (AIBN, Vazo 64™, DuPont) was added. Subsequently the external heating bath was heated to 75° C. and the reaction was carried out constantly at this external temperature. After a reaction time of 1 h a further 0.3 g of AIBN was added. Dilution took place after 4 h and 8 h, using 100 g of acetone each time. After a reaction time of 48 h the reaction was terminated and cooling took place to room temperature.

Subsequently the polymer was blended in solution with 8% by weight of bis[[(2-nitro-benzyl)oxy]carbonyl]hexane-1,6-diamine and then concentrated in a vacuum drying oven at 80° C. and a pressure of 10 torr. Subsequently the hotmelt process was carried out in the recording extruder. For crosslinking, the acrylate hotmelt was coated at 50 g/m² onto a PET film, using a heatable laboratory roll coater, lined with a siliconized PET film, masked using a mask having a mesh size of 60 μm and an open area of 40%, irradiated with UV light, and following removal of the mask conditioned subsequently at 120° C. for 10 minutes. Finally the specimens were tested according to methods A and B.

### Reference Example R1

A 2 L glass reactor conventional for radical polymerizations was charged with 40 g of acrylic acid, 360 g of 2-ethylhexyl acrylate, 0.55 g of bis-2,2′-phenylethyl thiocarbonate and 170 g of acetone. After nitrogen gas had been passed through the reactor for 45 minutes, with stirring, the reactor was heated to 58° C. and 0.3 g of azoisobutyronitrile (AIBN, Vazo 64™, DuPont) was added. Subsequently the external heating bath was heated to 75° C. and the reaction was carried out constantly at this external temperature. After a reaction time of 1 h a further 0.3 g of AIBN was added. Dilution took place after 4 h and 8 h, using 100 g of acetone each time. After a reaction time of 48 h the reaction was terminated and cooling took place to room temperature.

Subsequently the polymer was concentrated in a vacuum drying oven at 80° C. and a pressure of 10 torr. Subsequently the hotmelt process was carried out in the recording extruder. For crosslinking, the acrylate hotmelt was coated at 50 g/m² onto a PET film, using a heatable laboratory roll coater, irradiated with UV light in a number of passes, in accordance with the method described above, and then conditioned at 140° C. for 20 minutes. Finally the specimens were tested according to methods A and B.

Results

Set out below are the results of Examples 1 to 6. With bis[[(2-nitrobenzyl)oxy]carbonyl]-hexane-1,6-diamine (XIIa) and 4,4′-[bis[[(2-nitrobenzyl)oxy]carbonyl]trimethylene]-dipiperidine (XIIb), two different photobase generators were trialed. Additionally, different crosslinking mechanisms were trialed. In Examples 1 to 3 crosslinking took place via the carboxylic acid groups of the polyacrylate PSAs. In Example 4 crosslinking via epoxide groups was investigated, and in Example 5 crosslinking via isocyanate groups. In Example 6 the acrylate hotmelt PSA of the invention was irradiated selectively, through a mask, so as to generate a structured PSA by way of the photobase technique.

To investigate their suitability as hotmelt PSAs, all of the examples were subjected to a hotmelt process. Following polymerization and blending with the photobase generator, the solvent was removed and then the hotmelt PSA was kneaded in a hotmelt extruder at 120° C. for 6 hours. In none of Examples 1 to 6 was an increase in torque found during this period, which would have indicated gelling and hence thermal instability. To confirm this, the gel index was determined as well. The gel indices measured were all 0 and therefore additionally confirmed the thermal stability of the acrylate hotmelt PSAs.

In order to examine their suitability as PSAs the adhesive performance properties were likewise determined. In addition, a reference sample R1 was included, which contains exactly the same comonomer composition as Example 1 but without any photobase generator. Since the adhesive performance properties in particular are dependent on the successful crosslinking of the hotmelt PSAs, the table below likewise lists the UV irradiation doses in the UV-C and UV-B range.

TABLE 1

| | | | SWT 5 N, | |
|---|---|---|---|---|
| | | SWT 10 N, | 70° C. | |
| Ex- | UV dose | RT [min] | [min] | BS-steel [N/cm] |
| ample | [mJ/cm²] | (test A1) | (test A2) | (test B) |
| 1 | UV-C: 150 | +10000 | +10000 | 4.1 |
| | UV-B: 840 | | | |
| R1 | UV-C: 150 | 325 | 15 | 1.8 |
| | UV-B: 840 | | | |
| 2 | UV-C: 150 | +10000 | +10000 | 4.2 |
| | UV-B: 840 | | | |
| 3 | UV-C: 150 | 7685 | 4520 | 3.9 |
| | UV-B: 840 | | | |
| 4 | UV-C: 200 | 1240 | 2295 | 4.3 |
| | UV-B: 1120 | | | |
| 5 | UV-C: 200 | 1550 | 1155 | 4.2 |
| | UV-B: 1120 | | | |
| 6 | UV-C: 300 | 2475 | 3420 | 5.1 |
| | UV-B: 1680 | | | |

50 g/m² application rate to PET film
SWT: shear withstand times
BS: bond strength

A comparison of Examples 1 and R1 shows that as a result of adding the photobase generator the crosslinking behavior of the PSA is markedly improved. The cohesion (shear withstand times) of Example 1 are much higher. Moreover, in the course of the bond strength measurement, the reference example splits cohesively, so that here it can be assumed that the PSA tape has undergone virtually no crosslinking. Examples 2 to 5 establish the fact that different photobase generators can be used, that the cohesion of the PSA can be controlled ultimately by the comonomer composition of the acrylate hotmelt PSAs of the invention, and that different functional groups can be used for crosslinking. Example 6 affords the possibility of producing structured PSAs too by means of targeted crosslinking via the PSAs of the invention.

1. A polyacrylate pressure-sensitive adhesive comprising a polymer formed from
a) a comonomer mixture comprising
a1) 55%-99% by weight, based on component (a), of acrylic acid and/or acrylic esters of the formula:
CH₂═C(R¹)(COOR²),
where R¹═H or CH₃ and R² is an alkyl chain having 1-20 carbon atoms,
a2) 0-30% by weight, based on component (a), of olefinically unsaturated monomers having functional, aromatic, heteroaromatic or heterocyclic groups, or a combination of any of said groups, wherein said functional groups are selected from the group consisting of hydroxyl groups, sulfonic acid groups, ester groups, ether groups, anhydride groups, epoxy groups, amide groups, and amino groups,
a3) 1%-15% by weight, based on component (a), of acrylate or methacrylate monomers having at least one functional group, which is capable of reacting with a photochemically generated base b), with or without addition of a catalyst,
the polymer being thermally crosslinked at least partly with 0.01%-25% by weight, based on the overall polymer mixture, of a base b).

2. The polyacrylate pressure-sensitive adhesive of claim 1, wherein the monomers a1) are selected from the group consisting of acrylic monomers which comprise acrylic and methacrylic esters having alkyl groups consisting of 4 to 14 carbon atoms.

3. The polyacrylate pressure-sensitive adhesive of claim 1, wherein the monomers a3) are selected from the group consisting of comonomers containing at least one carboxylic acid group, one isocyanato group or one epoxide group.

4. The polyacrylate pressure-sensitive adhesive of claim 1, wherein said photochemically generated base is generated from a photobase generator selected from the group consisting of O-acyl oximes, anilide derivatives, ammonium salts and organometallic compounds which liberate a base under UV irradiation.

5. A process for preparing polyacrylate hotmelt pressure-sensitive adhesives from polymers formed from
a) a comonomer mixture comprising
a1) 55%-99% by weight, based on component (a), of acrylic acid and/or acrylic esters of the formula:
CH₂═C(R′)(COOR²),
where R¹═H or CH₃ and R² is an alkyl chain having 1-20 carbon atoms,
a2) 0-30% by weight, based on component (a), of olefinically unsaturated monomers having functional groups selected from the group consisting of hydroxyl groups, sulfonic acid groups, ester groups, ether groups, anhydride groups, epoxy groups, amide groups and amino groups, or having aromatic, heteroaromatic and/or heterocyclic groups,
a3) 1%-15% by weight, based on component (a), of acrylate or methacrylate monomers having at least one functional group which is capable of reacting with the base generated by a photobase generator b), with or without a catalyzing compound, and
b) 0.01%-25% by weight, based on the overall comonomer mixture, of at least one photobase generator b
where said photobase generator b) is incorporated into the comonomer mixture by mixing or copolymerization and where the solvent-free polymer or the polymer substantially freed from solvent, with the photobase generator incorporated therein, is coated, in a hotmelt process, onto a backing, and during or after coating is irradiated with UV light, thereby generating a base photochemically, and the composition is subsequently crosslinked thermally by the reaction of at least component a3) with the base.

6. The process of claim 5, wherein solvent, if present, is removed with heating under reduced pressure.

7. The process of claim 5, wherein the polymer is placed onto a film of water, with subsequent transfer from the film of water to the backing material, the water optionally contributing to the crosslinking of the pressure-sensitive adhesive.

8. The process of claim 5, wherein said UV irradiation takes place during coating.

9. The process of claim 5, wherein the polyacrylate pressure-sensitive adhesive on the backing material is irradiated with UV light over its full area and subsequently heated, for the purpose of thermal crosslinking, to a temperature of at least 80° C.

10. The process of claim 5, wherein structured polyacrylates are prepared by performing a structured crosslinking by irradiating the polymer coating with ultraviolet light in such a way that only certain regions of the polymer mixture are exposed to the UV radiation.

11. The process of claim 10, wherein the polymer coating is irradiated with ultraviolet light through a perforated mask.

12. The process of claim 10, wherein the polymer coating is irradiated with ultraviolet light through a film whose surface has regions of different UV light transparency, whereby certain regions of the polymer mixture are exposed to different intensities of UV radiation.

13. Pressure-sensitive adhesive tapes and strips coated on one or both sides with the polyacrylate pressure-sensitive adhesive of claim 1.

14. The pressure-sensitive adhesive of claim 2, wherein said alkyl groups consist of 4-9 carbon atoms.

15. The pressure sensitive adhesive of claim 14, wherein said monomers a1) are selected from the group consisting of n-butyl acrylate, n-pentyl acrylate, n-hexyl acrylate, n-heptyl acrylate, n-octyl acrylate, n-nonyl acrylate, lauryl acrylate, stearyl acrylate, behenyl acrylate, and their branched isomers.

16. The polyacrylate pressure-sensitive adhesive of claim 3, wherein the monomers a3) are selected from the group consisting of glycidyl methacrylate, acrylic acid, methacrylic acid and 2-isocyanatoethyl methacrylate.

17. The process of claim 9, wherein said temperature is up to about 100° C.
